(19) United States
(12) Patent Application Publication
(10) Pub. No.: US 2022/0015973 A1
(43) Pub. Date: Jan. 20, 2022

(54) Intubation Infection Control Tent

(71) Applicant: S.W.O.R.D. International Inc., Sparks, NV (US)

(72) Inventors: Christopher Collin Klaftenegger, Reno, NV (US); Jeremy Allen Elrod, Reno, NV (US)

(21) Appl. No.: 17/377,208

(22) Filed: Jul. 15, 2021

Related U.S. Application Data
(60) Provisional application No. 63/052,287, filed on Jul. 15, 2020.

(51) Int. Cl. A61G 10/00; A61M 16/00

(52) U.S. Cl. CPC A61G 10/005; A61M 2202/0208; A61M 16/0003

(57) ABSTRACT

An intubation infection control tent system is described. In an example implementation, an infection control tent includes a cover that forms a space in which a patient can be positioned, the cover creating an infectious barrier between the patient and an external environment, one or more support channels integrated into the cover that provide a rigid structure to the cover, and a drape skirt that connects to an edge of the cover and drapes over a portion of the patient.

### CROSS-REFERENCE TO RELATED APPLICATIONS

The present application also claims the benefit under 35 U.S.C. § 119(e) of U.S. Provisional Patent Application Ser. No. 63/052,287, titled “Intubation Infection Control Tent”, filed on July 15, 2021, the entire contents of which are incorporated herein by reference.

### BACKGROUND

The present disclosure relates to an intubation infection control tent system and methods of using the intubation infection control tent system.

Highly infection diseases, such as Ebola, H1N1 Influenza, Hantavirus, or COVID 19, to name a few have been making their way into society. These infections diseases are airborne as well as surface-borne and have had a devastating effect on first responders and medical treatment personnel as the go about assisting in the treatment of these diseases. It is known that hazmat suits can protect these individuals, but the use of such suits can seriously impede the quality and speed of treatment for patients in immediate need of medical assistance

### SUMMARY

According to one innovative aspect of the subject matter in this disclosure, an intubation infection control tent system is described. In an example implementation, one general aspect includes an infection control tent that also includes a cover that forms a space in which a patient can be positioned, the cover creating an infectious barrier between the patient and an external environment; one or more support channels integrated into the cover that provide a rigid structure to the cover, and a drape skirt that connects to an edge of the cover and drapes over a portion of the patient.

Implementations may include one or more of the following features. The infection control tent where the one or more support channels are inflatable and provide the rigid structure when the one or more support channels are inflated and the cover is in an unfolded configuration. The cover is configured to be in a folded configuration when the one or more support channels are deflated. The drape skirt includes a drape skirt support channel that extends along a bottom edge of the drape skirt. The cover includes one or more hand insertion ports that allow a medical provider to access the patient within the cover. The one or more hand insertion ports each include an inner overlapping material to seal the one or more hand insertion ports. The cover includes one or more gas tube exchange ports that control a positive pressure and a negative pressure within cover. The one or more gas tube exchange ports include a first gas tube exchange port and a second gas tube exchange port and where the first gas tube exchange port is connected to a positive pressure connection and the second gas tube exchange port is connected to a negative pressure connection. The negative pressure connection uses evacuation pressure to remove infectious aerosol from the cover. The positive pressure connection is connected to oxygen. The drape skirt is configured to be lifted to pass equipment into an area within the cover. The cover is formed out of a see-through plastic. The support channels are one of a plastic framing and a metal spring wire. The cover is further supported by one or more support rods that brace against the support channels.

One general aspect includes an infection control tent that also includes a cover formed out of a see-through plastic, the cover including a substantially flat bottom surface and a cylindrical side wall that connects to opposite sides of the bottom surface and forms a space in which a patient can be positioned; a supporting channel integrated into the cylindrical side wall that provides a rigid structure to preserve a cylindrical shape of the cylindrical sidewall, a top surface that connects along a top edge of the cylindrical side wall and along a top edge of the bottom surface, and a drape skirt that connects along a bottom edge of the cylindrical side wall and drapes down over the patient when the patient is positioned within the space.

Implementations may include one or more of the following features. The infection control tent where the supporting channel is an inflatable channel that provides the rigid structure when inflated. The infection control tent may include: a gas tube exchange port integrated into the cylindrical side wall that allows for pressure changes to be controlled within the space in which the patient can be positioned. The drape skirt includes a drape skirt supporting channel that maintains a shape of the drape skirt.

One general aspect includes a foldable infection control tent that also includes a cover including a cylindrical side-wall that connects to a bottom surface, the cover being foldable into a folded configuration and an unfolded configuration, the cover forming a space in which a patient can be positioned in the unfolded configuration; and a supporting channel, the supporting channel being configured to fold down in the folded configuration and provide a rigid structure to the cover in the unfolded configuration.

Implementations may include one or more of the following features. The foldable infection control tent where the supporting channel is inflatable and wherein the supporting channel is deflated in the folded configuration and is inflated to provide the rigid structure in the unfolded configuration.

The above and other implementations are advantageous in a number of respects as articulated through this document. Moreover, it should be understood that the language used in the present disclosure has been principally selected for readability and instructional purposes, and not to limit the scope of the subject matter disclosed herein.

### BRIEF DESCRIPTION OF THE DRAWINGS

The disclosure is illustrated by way of example, and not by way of limitation in the figures of the accompanying drawings in which like reference numerals are used to refer to similar elements.

FIG. 1 is an example implementation of an intubation infection control tent system.

FIG. 2 is a perspective view of an example intubation infection control tent system.

FIG. 3 is a side view of an example intubation infection control tent system.

FIG. 4 is a top view of an example intubation infection control tent system.

FIG. 5 is a front view of an example intubation infection control tent system.

FIG. 6 is a bottom view of an example intubation infection control tent system.

FIG. 7 is a close-up view of the connection area of a cover and drape skirt of an implementation of an intubation infection control tent system.

### DETAILED DESCRIPTION

The technology described herein provides an intubation infection control tent system. In addition to the described technology, other technologies are also described and contemplated herein. The intubation infection control tent system 100 may provide a mechanism for patient treatments that isolates first responders and/or medical treatment personnel from infection media, while at the same time allowing those persons to treat the patient in a rapid and effective manner. The intubation infection control tent system 100 provides a patient ventilation and isolation shield for intubation and treatment without exposing first responders or medical staff to potential infections media.

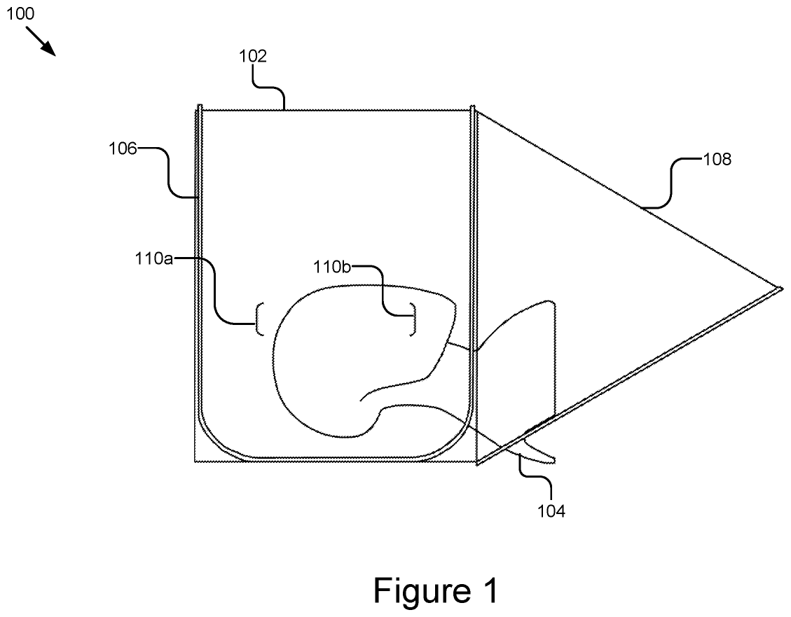

FIG. 1 is an example implementation of an intubation infection control tent system 100. As shown in FIG. 1, the intubation infection control tent system 100 may include a cover 102 that is configured to surround at least a portion of a patient 104. The cover 102 may form a space in which a patient 104 can be positioned that creates an infectious barrier between the patient 104 and an external environment. The cover 102 allows for isolation of the patient 104 by acting as a barrier between the patient 104 and an air environment and/or surface environment of a treatment location for the patient 104. This barrier prevents any infection media from affecting first responders or other medical professionals without the need of having the first responders and/or medical professionals covering themselves in Hazmat Suits or other protective equipment.

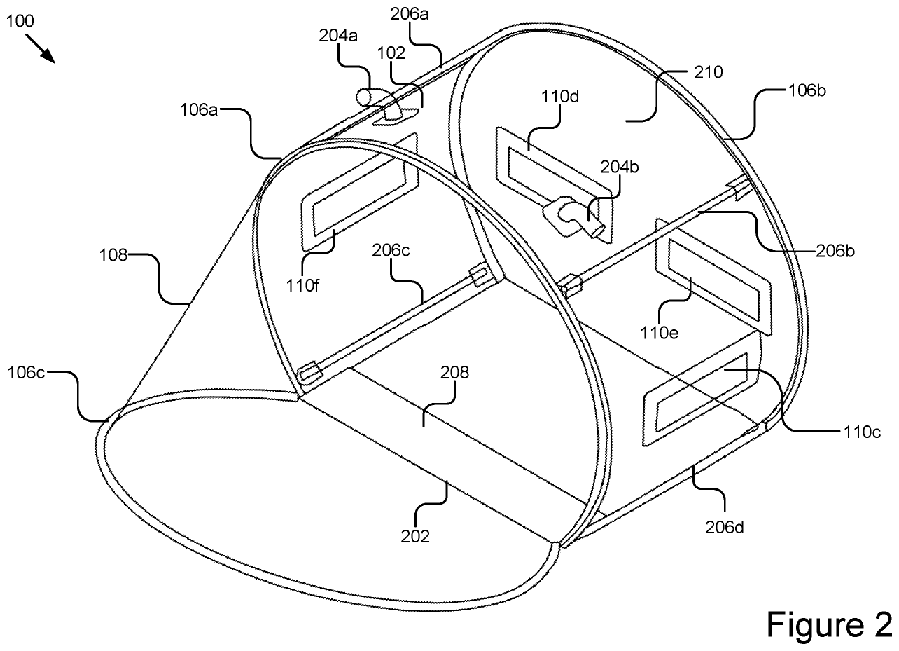

The cover 102 may be configured to wrap around a portion of a patient's 104 head and chest. In some implementations, the cover 102 may be substantially cylindrical in shape and may curve from one side of a patient's 104 to the other. The cover 102 may be formed out of a see-through material such as a see-through plastic and may include a cylindrically shaped side-wall that wraps around the patient 104 and connects on opposite sides to a bottom surface to form the space in which the patient is positioned. In further implementations, other shapes of the cover 102 are contemplated, such as a square rectangle. In some implementations, as discussed in more detail with respect to FIG. 2, the cover 102 may have a bottom surface that rests underneath a patient 104, and the bottom surface slides under a portion of the patient's 104 head. The bottom surface may connect to the edge of the cover on both sides of the patient's 104 head and the cover my extend upward from the bottom surface and curve around the front of the patient's 104 head to form a space within the intubation infection control tent system 100 that creates a barrier for infection control between the patient 104 on the interior of the cover 102 and others on the exterior of the cover 102. In some implementations, the cover 102 may also include a top surface that connects to the top edge of the cylindrical side-wall of the cover 102 and the bottom surface to create a barrier along the top of the patient's 104 head.

In some implementations, the cover 102 may connect along an edge opposite the edge, such as a bottom edge of the cylindrical side wall, connected to the top surface of the cover 102 to a drape skirt 108. The drape skirt 108 may be configured to rest or drape over at least a portion of a patient's 104 body and rest on a patient's 104 chest. In further implementations, the drape skirt 108 may cover the entire body of the patient 104. In some implementations, the shape of the drape skirt 108 may be shaped using a drape skirt support channel 106c, similar to the other support channels 106 as described elsewhere herein. The drape skirt support channel 106c may maintain the drape skirt 108 shape. In further implementations, the drape skirt support channel 106c may be easily lifted, such as with a single hand to provide access to and/or pass equipment into the space within the cover 102. In some implementations, one or more sides of the cover 102 may include one or more hand insertion ports 110 that a medical professional may use to interact with the patient 104.

In some implementations, the cover 102 may be supported by one or more support channels 106 (also referred to herein as 106a . . . 106n). The support channels 106 may be configured to provide a rigid support to keep the structure of the cover 102 in place as a barrier and create a space within the cover 102 for the patient 104. In some implementations, the support channels 106 may be integrated into the cylindrical side wall of the cover 102 to provide the rigid structure and preserve a cylindrical shape of the cylindrical sidewall.

In some implementations, the intubation infection control tent system 100 may be configured to fold up when not in use. In the folded configuration, the support channels 106 may fold in on themselves to allow the cover 102 and/or drape skirt 108 to fold and flatten down for storage. When the intubation infection control tent system 100 is needed, the support channels 106 can be positioned to give form to the cover 102 and create a barrier that can be rapidly deployed and positioned around a patient 104. The foldable nature of the infection control tent allows the intubation infection control tent system 100 to be stored in a folded configuration that is small and portable, which also allows for large quantities of the intubation infection control tent system 100 to be stored and moved in the folded configuration. When the intubation infection control tent system 100 is needed, the support channels 106 can be positioned to form the space on the interior of the cover 102 and create a barrier in the unfolded position that can then be used with a patient 104. This rapid deployment option makes the intubation infection control tent system 100 an ideal piece of equipment for first responders that may not know if a patient is infection and may not be able to carry large amounts of protective equipment, but can still protect themselves from infectious transmission by deploying the intubation infection control tent system 100 when they arrive on the scene.

FIG. 2 is a perspective view of an example intubation infection control tent system 100. As shown in FIG. 2, the cover 102 may be formed out of a bottom surface 202, a top surface 210, and a drape skirt 108, that are all connected along the edges of the cover 102 to form the barrier between a patient 104 (not shown) and the external environment. In some implementations, the cover 102 forms a box with one or more support channels 106 to provide rigidity when in the unfolded position. In some implementations, the support channels 106 (such as 106a connected between the edge of the cover 102 and the drape skirt 108 or 106b connected between the edge of the cover 102 and the top surface 210) may be configured to fold and unfold for rapid deployment. These two different configuration may be achieved using support channels formed out of metal spring wire or plastic framing that allows the cover 102 to fold in on itself in the folded configuration.

In some implementations, the support channels 106 may be inflatable tubes designed to inflate and create a rigid support structure when in the unfolded state. In some implementations, these inflatable tubes may be conjoined or integrated into the cover 102 and provide rigid structure when inflated. In further configurations, the inflatable tubes may be separate from the cover 102 and the cover may be attachably connected to the inflatable tubes when they are inflated to form the rigid support structure. In some implementations, the inflatable tubes may have a connection point for inflation by an inflation mechanism, such as oral inflation, manual pump, electric pump, pressure actuator, or a gas cartridge containing carbon dioxide, nitrogen, or another compressed gas for inflation. By using the inflatable tubes to create the rigid support structure, the barrier between the patient 104 and the external environment is capable of being formed/inflated as the cover 102 is being deployed over the patient 104.

In some implementations, the cover 102 may be further supported using one or more support rods 206, such as 206a-206d. These support rods 206 may provide additional rigid structure to the cover 102. In some implementations, the ends of the support rods 206 may be configured to slide into pockets formed into the exterior (or interior) of the cover 102 and brace the support channels 106. In some implementations, the support rods 206 may be rigid rods that are designed to fold up with the cover in the folded configuration and then be quickly positioned in place within the pockets during the unfolded deployment of the cover 102. In further implementations, other attachment mechanisms may be used to connect the support rods 206 to the cover 102.

In some implementations, the cover 102 and/or other components, such as the top surface 210, bottom surface 202, and/or drape skirt 108 of the intubation infection control tent system 100 may be at least partially see-through to allow for viewing of the patient 104 within the intubation infection control tent system 100. In some implementations, the cover 102 and/or other components, such as the top surface 210, bottom surface 202, and/or drape skirt 108 of the intubation infection control tent system 100 may be formed out of a see-through plastic that creates a barrier for infectious transmissions while also allowing viewing into or out of the intubation infection control tent system 100.

In some implementations, the cover 102 may include one or more hand insert holes 110, such as 110c-110f in FIG. 2. These hand insert holes allow a medical provider to gain access to the patient 104 while the medical provider is still being isolated from any infection media associated with the patient. The hand insert holes 110 may be formed out of the material of the cover 102 and/or top surface 210 with an inner overlapping material that provides closure when a hand is not inserted into the hand insert hole 110. The inner overlapping material may create a seal between the inner overlapping material and the material of the cover 102. The hand insert holes 110 may designed and positioned anywhere along the intubation infection control tent system 100 in order to make it easier to treat the patient 104, such as along the sides and/or top of the intubation infection control tent system 100.

In some implementations, the drape skirt 108 may also include a drape skirt support channel 106c along the edge that is configured to rest on a patient's 104 chest. This support channel 106c allows the drape skirt to be lifted using a single hand or automatically lifted if connected to a machine and equipment can be passed through to the patient 104 when the drape skirt 108 is lifted. The drape skirt 108 is designed to cover the patient 104 from below the chest and the support channel 106c rests on a portion of the patient's 104 chest.

In some implementations, the intubation infection control tent system 100 may use one or more of positive and/or negative pressure to provide ventilation for patients 104 with breathing difficulties. One or more gas tube exchange ports 204 may be positioned within the material of the cover 102 and allow for pressure changes to be controlled within the space in which the patient is positioned. In some implementations, the gas tube exchange ports 204 may include an inner overlapping material to provide closure and seal the gas tube exchange port 204 closed. In some configurations, a positive pressure connection may be made to a first gas tube exchange port 204 that provides positive pressure, such as by providing oxygen through the gas tube exchange port 204. In some configurations, a negative pressure connection may be made to a second gas tube exchange port 204, such as by providing suction through the gas tube exchange port 204. In some implementations, these positive pressure connections and/or negative pressure connections can be used to remove infectious aerosol virus from the intubation infection control tent system 100.

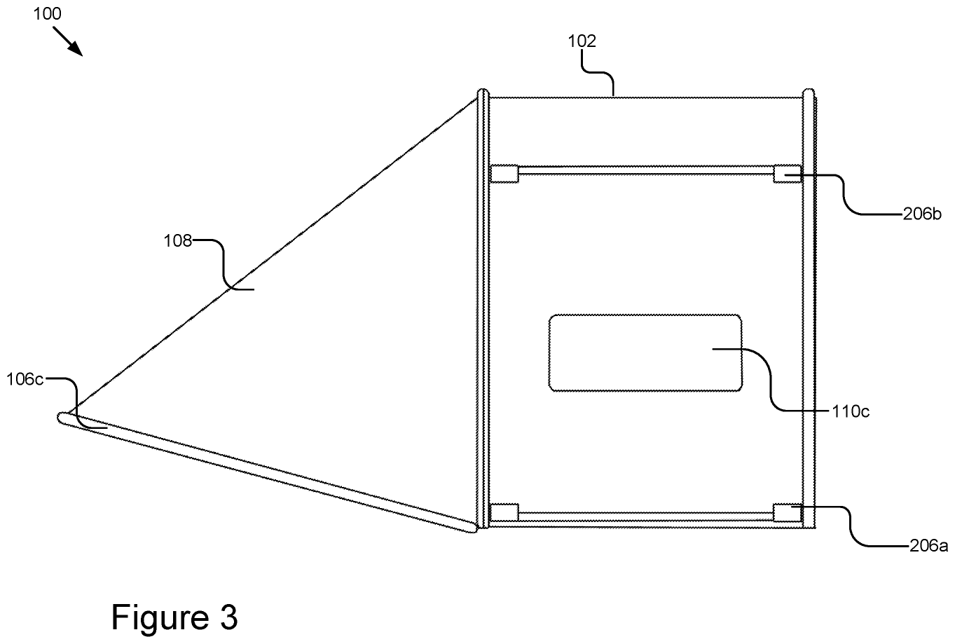

FIG. 3 is a side view of an example intubation infection control tent system 100. As shown in FIG. 3, the drape skirt 108 may be configured to rest in an angled-up from the bottom edge of the cover 102 to a raised position to allow the support channel 106c to rest on a patient's 104 chest. As shown, a side hand insert hole 110c may be positioned in a side-wall, such as a cylindrical side-wall, of the cover 102.

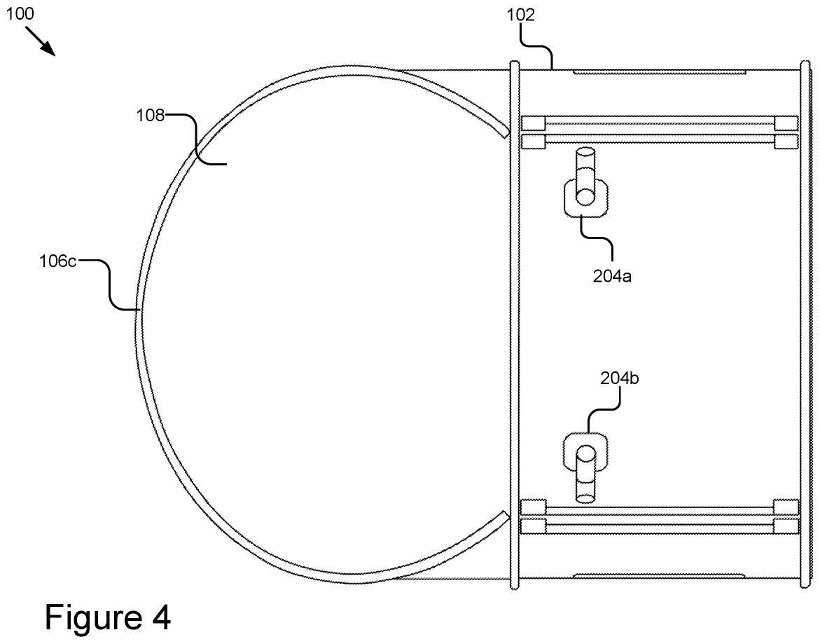

FIG. 4 is a top view of an example intubation infection control tent system 100. As shown in FIG. 4, the gas tube exchange ports 204 may be positioned along a top surface of the cover 102, although other locations are also contemplated. As shown, the cover 102 may form a substantially cylindrical shape to form a space of the patient 104 within the cover 102. In some implementations, the top surface is substantially flat to assist when positioning in various environments, such as hospital beds, vehicles, etc. although other configurations are also contemplated.

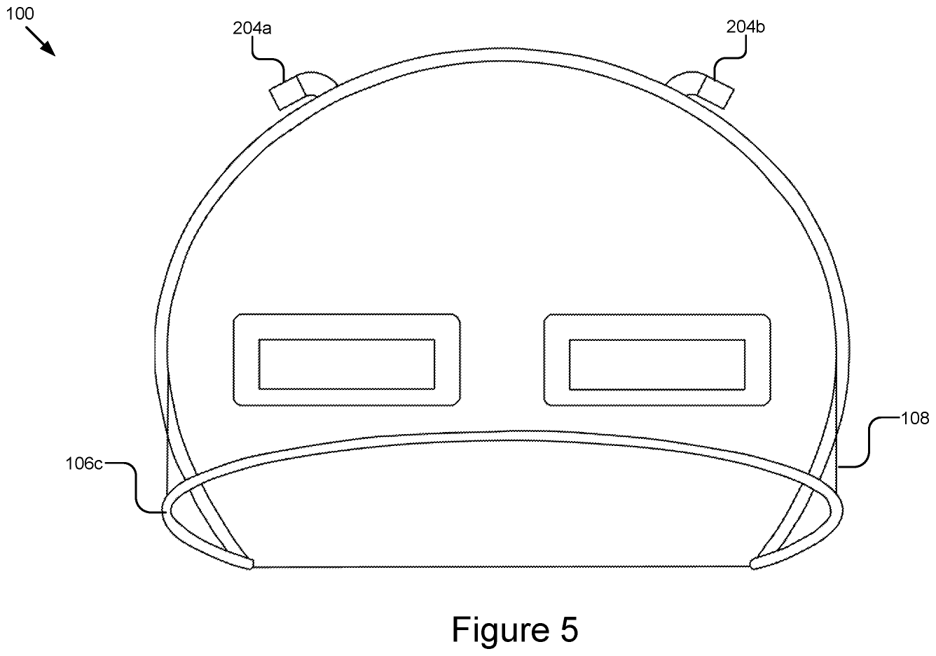

FIG. 5 is a front view of an example intubation infection control tent system 100. As shown in FIG. 5, the support channel 106c may be slightly raised when rigid to allow for a patient's 104 body to extend underneath the drape skirt 108. As shown, the support channel 106c may wrap around a bottom edge of the drape skirt 108 and connect at each corner of the front edge of the cover 102 along the bottom edge. This creates a point for the support channel 106c to rotate along when the support channel 106c is lifted.

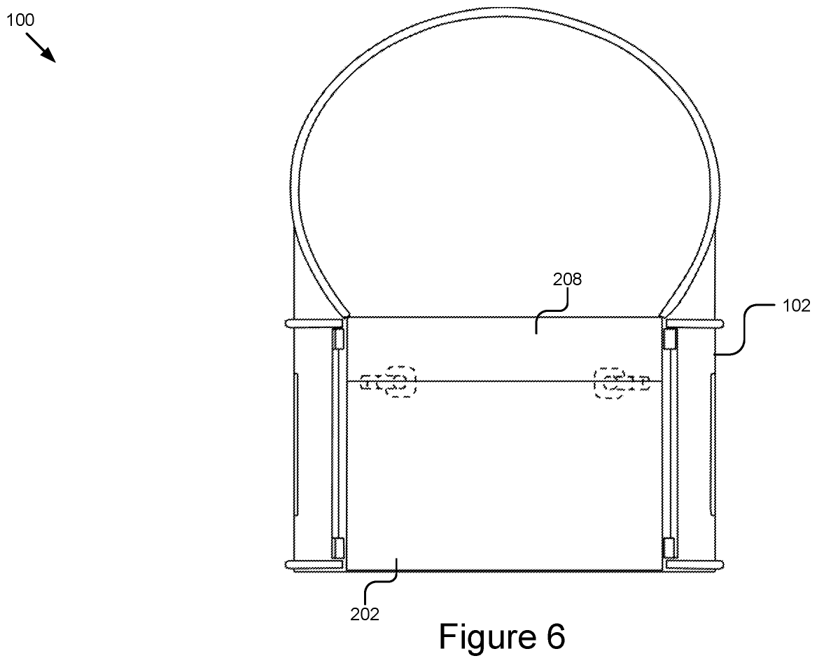

FIG. 6 is a bottom view of an example intubation infection control tent system 100. As shown in FIG. 6, a bottom surface 202 may be substantially flat and may be designed to slid under a patient 104. In some implementations, the bottom surface 202 may be folded over itself to form a pocket 208 along the bottom surface 202 where the bottom surface 202 connects with the drape skirt 108 along a front edge of the cover 102.

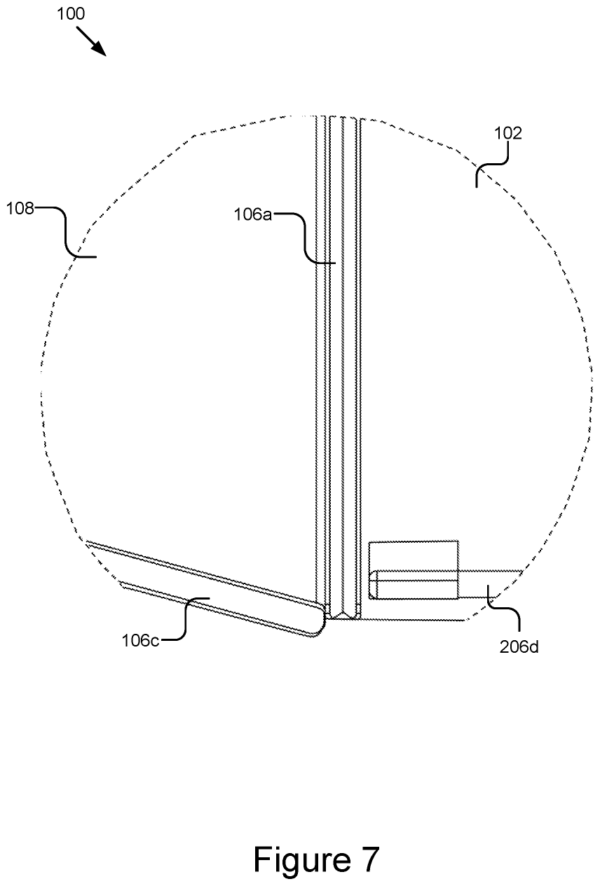

FIG. 7 is a close-up view of the connection area of a cover and drape skirt of an implementation of an intubation infection control tent system 100. As shown in FIG. 7, the support channel 106a connects to the cover 102 along an edge and the support channel 106c of the drape skirt 108 may be attached/formed into the material at the support channel 106a for rotation as described elsewhere herein. In some implementations, the shape of the cover 102 may have relief curves that allow for the device to be deployed to a point on the patient's 104 chest below the neck while still effectively covering the patient's 104 head, face, and neck.

In some implementations, the intubation infection control tent system 100 may be equipped with LED lighting, such as lights attached along or incorporated into one or more support channels 106 to provide light to the patient. In some implementations, the LED lighting may be solar, or battery powered to assist in operations in the field. In some implementations, the intubation infection control tent system 100 may include one or more UV disinfection lights positioned to shine within the cover 102 and provide disinfection along with protection. In some implementations, the intubation infection control tent system 100 may include one or more disposable glove inserts, such as attached to or in replacement of the hand insertion ports 110 to provide additional protection to the medical provider. In some implementations, an anti-viral filter may be attached to the negative pressure connection of the gas tube exchange port 204 to filter out one or more of the infection material within the cover 102. In some implementations, the intubation infection control tent system 100 may include a hardened intubation shield that may be integrated into the device, such as for Emergency Room, ICU, and/or other surgery austere environments.

In some implementations, the intubation infection control tent system 100 may be designed to use a low-cost plastic sheeting with one or more of wire inserts or inflatable tubes for the support channels 106. Using the low-cost plastic sheeting and/or other low-cost materials allows for rapid and cheap manufacturing compared to other protective material being using by healthcare professionals currently. This allows for a low-cost the intubation infection control tent system 100 to be manufactured and the foldable design allows for large amounts of the intubation infection control tent system 100 to be easily packaged and shipped as needed.

The intubation infection control tent system 100 provides rapid treatment protocols can be instituted on a patient 104 without first requiring treating personnel to cover themselves in Hazmat suits or other protective equipment. Additionally, it provides treating personnel, such as first responders, with protective measures when additional protective equipment may not be available, such as when traveling to a patient locations, such as an accident scene, etc. For medical personnel, the intubation infection control tent system 100 allow for a rapid isolation event to take place when the intubation infection control tent system 100 is deployed in the unfolded state. This rapid isolation event prevents significant exposure of the air environment and surface environment of the treatment location to the infectious media. The intubation infection control tent system 100 can be used in hospitals, emergency treatment units, ambulance services, and other implementations.

It should be understood that the above-described example activities are provided by way of illustration and not limitation and that numerous additional use cases are contemplated and encompassed by the present disclosure. In the above description, for purposes of explanation, numerous specific details are set forth in order to provide a thorough understanding of the present disclosure. However, it should be understood that the technology described herein may be practiced without these specific details.

The foregoing description has been presented for the purposes of illustration and description. It is not intended to be exhaustive or to limit the specification to the precise form disclosed. Many modifications and variations are possible in light of the above teaching. It is intended that the scope of the disclosure be limited not by this detailed description, but rather by the claims of this application. As will be understood by those familiar with the art, the specification may be embodied in other specific forms without departing from the spirit or essential characteristics thereof. Accordingly, the disclosure is intended to be illustrative, but not limiting, of the scope of the subject matter set forth in the following claims.

What is claimed is:

1. An infection control tent comprising:
a cover that forms a space in which a patient can be positioned, the cover creating an infectious barrier between the patient and an external environment;
one or more support channels integrated into the cover that provide a rigid structure to the cover; and
a drape skirt that connects to an edge of the cover and drapes over a portion of the patient.

2. The infection control tent of claim 1, wherein the one or more support channels are inflatable and provide the rigid structure when the one or more support channels are inflated and the cover is in an unfolded configuration.

3. The infection control tent of claim 2, wherein the cover is configured to be in a folded configuration when the one or more support channels are deflated.

4. The infection control tent of claim 1, wherein the drape skirt includes a drape skirt support channel that extends along a bottom edge of the drape skirt.

5. The infection control tent of claim 1, wherein the cover includes one or more hand insertion ports that allow a medical provider to access the patient within the cover.

6. The infection control tent of claim 5, wherein the one or more hand insertion ports each include an inner overlapping material to seal the one or more hand insertion ports.

7. The infection control tent of claim 1, wherein the cover includes one or more gas tube exchange ports that control a positive pressure and a negative pressure within cover.

8. The infection control tent of claim 7, wherein the one or more gas tube exchange ports include a first gas tube exchange port and a second gas tube exchange port and wherein the first gas tube exchange port is connected to a positive pressure connection and the second gas tube exchange port is connected to a negative pressure connection.

9. The infection control tent of claim 8, wherein the negative pressure connection uses evacuation pressure to remove infectious aerosol from the cover.

10. The infection control tent of claim 8, wherein the positive pressure connection is connected to oxygen.

11. The infection control tent of claim 1 wherein the drape skirt is configured to be lifted to pass equipment into an area within the cover.

12. The infection control tent of claim 1, wherein the cover is formed out of a see- through plastic.

13. The infection control tent of claim 1, wherein the support channels are one of a plastic framing and a metal spring wire.

14. The infection control tent of claim 1, wherein the cover is further supported by one or more support rods that brace against the support channels.

15. An infection control tent comprising:
a cover formed out of a see-through plastic, the cover including a substantially flat bottom surface and a cylindrical side wall that connects to opposite sides of the bottom surface and forms a space in which a patient can be positioned;
a supporting channel integrated into the cylindrical side wall that provides a rigid structure to preserve a cylindrical shape of the cylindrical sidewall;
a top surface that connects along a top edge of the cylindrical side wall and along a top edge of the bottom surface; and
a drape skirt that connects along a bottom edge of the cylindrical side wall and drapes down over the patient when the patient is positioned within the space.

16. The infection control tent of claim 15, wherein the supporting channel is an inflatable channel that provides the rigid structure when inflated.

17. The infection control tent of claim 15, further comprising:
a gas tube exchange port integrated into the cylindrical side wall that allows for pressure changes to be controlled within the space in which the patient can be positioned.

18. The infection control tent of claim 15, wherein the drape skirt includes a drape skirt supporting channel that maintains a shape of the drape skirt.

19. A foldable infection control tent comprising:
a cover including a cylindrical side-wall that connects to a bottom surface, the cover being foldable into a folded configuration and an unfolded configuration, the cover forming a space in which a patient can be positioned in the unfolded configuration; and
a supporting channel, the supporting channel being configured to fold down in the folded configuration and provide a rigid structure to the cover in the unfolded configuration.

20. The foldable infection control tent of claim 19, wherein the supporting channel is inflatable and wherein the supporting channel is deflated in the folded configuration and is inflated to provide the rigid structure in the unfolded configuration.
